(19) United States
(12) Patent Application Publication
(10) Pub. No.: US 2023/0363897 A1
(43) Pub. Date: Nov. 16, 2023

(54) BIOHYBRID HEART VALVE REPLACEMENT

(71) Applicant: THE TRUSTEES OF COLUMBIA UNIVERSITY IN THE CITY OF NEW YORK, New York, NY (US)

(72) Inventor: David KALFA, Long Island City, NY (US)

(21) Appl. No.: 17/849,565

(22) Filed: Jun. 24, 2022

Related U.S. Application Data
(63) Continuation of application No. PCT/US2020/067223, filed on Dec. 28, 2020.
(60) Provisional application No. 62/953,716, filed on Dec. 26, 2019.

(51) Int. Cl. A61F 2/24; D01F 6/62; D01F 6/70; D01D 5/00; B33Y 80/00

(52) U.S. Cl. CPC A61F 2/2418; A61F 2/2436; A61F 2/2415; D01F 6/625; D01F 6/70; D01D 5/003; B33Y 80/00; D01D 5/0084; A61F 2250/0082; D10B 2331/10; D10B 2331/041; D10B 2509/00; D10B 2401/12

(57) ABSTRACT

A heart valve replacement is provided including a tubular body portion having a proximal end, a distal end and a central portion arranged between said proximal and distal ends, defining a longitudinal direction of the valve replacement and having an inner wall region; a valve having at least one leaflet attached to the inner wall region of the central portion, each one of said leaflets being movable between a closing position and an opening position of the valve, wherein the tubular body portion is fabricated from a combination of a biostable polymer and a biodegradable biomaterial adapted to allow in-growth of tissue of the host and to increase its size concomitantly with surrounding organ structures of a host, and wherein the valve is fabricated of a biostable polymer connected to the biostable polymer of the tubular body portion.

### CROSS REFERENCE TO RELATED APPLICATIONS

This application is continuation of PCT/US2020/067223, entitled “Biohybrid Heart Valve Replacement”, filed Dec. 28, 2020 which claims priority to U.S. Provisional Application No. 62/953,716 filed Dec. 26, 2019, entitled “Heart Valve Replacement” all of which are incorporated by reference in their entirety herein.

### COPPYRIGHT NOTICE

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD

A biohybrid heart valve replacement designed to provide a living, growing, conduit and valve. More particularly, the heart valve replacement comprises a tubular body or conduit including biostable and biodegradable components that permit in-situ tissue regeneration and a growth compatible valve component.

### SUMMARY

In one aspect, a heart valve replacement that permits in-situ tissue regeneration and growth of the replacement is provided. The heart valve replacement comprises a tubular body having an inflow end, an outflow end and a generally cylindrical inner side wall portion extending between the inflow end and outflow end thereby forming a blood passage with an initial diameter. A valve defined by at least two leaflets is secured to an inner sidewall of the tubular body. Each leaflet is a longitudinal body comprising first and second opposing portions. The first portion of the leaflet is secured to the inner side wall portion of the tubular body and the second portion of the leaflet is a free edge configured to engage the corresponding second portion of an adjacent leaflet to close the valve. The inter-engaging portions of the leaflets are separable to open the valve, thus, the valve is configured to have a closed orientation and an open orientation. The tubular body is composed of material that permit in-situ tissue regeneration into the tubular body, such that the initial diameter of the tubular member increases over time after implantation. The material includes a combination of biostable and biodegradable polymers. Thus, the tubular body has a porosity pattern that becomes more porous as the biodegradable polymer degrades over time, thereby allowing replacement by living tissue and providing a growing vessel over time when implanted into a host, such as a child in need of a heart valve replacement. The heart valve replacement may be an aortic valve, tricuspid valve or mitral valve.

In some embodiments, the tubular body of the replacement is electrospun fibers comprising both biodegradable fibers and biostable fibers. In some embodiments, the biodegradable fibers are polycapriolactone (PCL), polyglycerol sebacate (PGS) or a combination of PCL and PGS. Generally, the ratio of PGS:PCL is between about 1:1 to 4:1. For example, but not limitation, in one embodiment, the ratio of PGS:PCL is about 3:1. The biostable fibers can be, for example, poly carbonate urethane (PCU).

In some embodiments, the biodegradable and biostable polymers are in the form of a mixture, or can be in a solution. In one embodiment, the tubular body comprises about 50 weight % polycarbonate urethane, 25 weight % PGS and 25 weight % PCL. In another embodiment, the tubular body comprises about 50 weight % PCU, 37.5 weight % PGS and 12.5 weight % PCL.

The valve is disposed in the conduit of the tubular body and provides for a growth compatible valve. The valve can be formed from non-porous, biostable polymeric material that does not degrade over time. The valve may have at least one or two leaflets. In some embodiments, the valve includes two or more leaflets each having sufficient height to maintain the competency of the valve while the initial diameter of the tubular body increases over time to a final diameter. For example, the initial diameter may be 12 mm and the final diameter 24 mm. In this manner, the leaflets may each have a height greater than the diameter of the tubular body. The leaflets may each have sufficient height of coaptation or sufficient length of the free edge to maintain competency of the valve while the diameter of the tubular body increases over time.

The valve can be secured to the tubular body, and in particular at a biostable region of the tubular body to form an integral heart valve replacement structure. In one embodiment, the at least two leaflets are sintered to the inner wall of the tubular body to form a superior robust connection with the tubular body. In this regard, the replacement may be sutureless.

In another aspect, a method of fabricating a heart valve replacement device is provided. The method provides a valved tube having a valve fully biostable that will remain inert, a porous tube made of a mix of bioresorbable and biostable polymers that will be replaced by a autologous living and growing tissue after implantation over time, and a mechanically robust cohesion between the valve and the tube after degradation of part of the tube. In accordance with one embodiment, the method includes preparing a valve comprising a first biostable polymer on a mandrel, preparing an electrospinning mixture the first biostable polymer and biodegradable polymers, and electrospinning the electrospinning mixture of polymers onto the mandrel to form an interconnected porous tubular body, such that there is continuity between the first biostable polymers present in the valve and the tubular body. In this regard, the valve may be prepared on the mandrel by dip molding, 3D printing or other techniques. The valve is non-porous, while the tubular body is porous and formed from electrospun fibers. The porosity pattern of the tubular member permits the penetration of autologous living and growing tissue to penetrate the interstices in the porous tubular body, as well as replace degrading biodegradable polymer over time. Thus, the heart valve replacement is a growing vessel capable of growing in situ after implantation into a patient.

In yet another aspect, a method of replacing a heart valve in a host, comprising the steps of: inserting a distal end portion of a delivery sheath into a portion of a heart of a host, the delivery sheath having a heart valve replacement according to any one of embodiments described and claimed herein is disposed within a lumen of the delivery sheath. The heart valve replacement is moved distally out of the delivery sheath and positioning the heart valve replacement within the heart of the host. The method may be for the treatment of aortic stenosis, mitral valve stenosis, regurgitation, or tricuspid valve regurgitation in the host. The host may be a child, for example, a child under the age of eighteen years old.

### BRIEF DESCRIPTION OF THE FIGURES

FIG. 1 is a schematic illustration of the heart valve replacement in accordance with the subject matter disclosed;

FIG. 2A is a top perspective of the heart valve replacement showing the valve in a closed position, and FIG. 2B shows the valve in an open position, in accordance with the subject matter disclosed;

FIG. 3 is a schematic illustration of a cross section of the tubular member and at least one leaflet in accordance with the disclosed subject matter;

FIG. 4 a schematic illustration of the growth compatible valve in accordance with the disclosed subject matter;

FIG. 5A to 5C shows stress strain curves for various mixtures of PCU and ratios of PGS:PCL tested in in accordance with the heart valve replacement disclosed;

FIGS. 6A to 6G illustrate surface porosity increasing as a matter of degradation time in a casted scaffold as compared to PCU in accordance with the disclosed subject matter;

FIG. 7A and 7B are bar graphs showing average pore radius and relative frequency of pore radius of study of FIG. 6A-6G in accordance with the disclosed subject matter;

FIG. 8A to FIG. 8B shows results of a degradation study depicting the impact of the PGL material used to fabricate the tubular body of the replacement in accordance with the disclosed subject matter;

FIG. 9 shows results of an in vitro the cell adhesion and proliferation study of a casted scaffold in accordance with the disclosed subject matter;

FIG. 10 shows results of an in vitro cell penetration study of a casted scaffold in accordance with the disclosed subject matter;

FIG. 11 shows extracellular matrix formation in an in vitro study of a casted scaffold in accordance with the disclosed subject matter;

FIG. 12 shows a comparison of porosity patterns in a casted tubular member and electrospun tubular member in accordance with the disclosed subject matter;

FIG. 13 shows a comparison of cell penetration after 7 days in a casted tubular member and electrospun tubular member in accordance with the disclosed subject matter; and

FIG. 14A-14E shows an exemplary method for fabricating the heart valve replacement in accordance with the disclosed subject matter; and

FIG. 15 shows mechanical testing results of the impact of PGL on the cohesion between the valve and tubular body in accordance with the disclosed subject matter.

### DETAILED DESCRIPTION OF EXEMPLARY EMBODIMENTS

In one aspect, a hybrid tissue-engineered heart valve replacement is provided that is particularly useful in pediatric applications, in that it is able to expand in size while the child grows, avoiding multiple reoperations. The replacement (or prosthesis) can be implanted surgically and is capable of growing with the child until the child reaches adulthood.

The heart valve replacement is a regenerative medicine-based device that includes a biohybrid (i.e., biostable and biodegradable polymer) tubular body and a growth-compatible polymeric valve. The heart valve replacement comprises a cylindrical tubular body and a valve component. The valve is made of a biostable polymer, and the tubular body is made of a blend or mixture of biostable polymer and biodegradable polymer. Thus, the tubular body has a porosity that increases as the biodegradable polymer degrades over time after implantation. The increase in porosity permits living tissue to replace the degrading polymer in the tubular body, thereby providing a replacement that grows over time, as the host grows. The host for example is a child under the age of eighteen years. As one of ordinary skill in the art would appreciate, all of the polymers utilized to manufacture the heart valve replacement may be biocompatible and in current use for clinical devices.

In some embodiments, the biodegradable polymer used as a component of the tubular body is combination of polyglycerol sebacate (PGS) and polycaprolactone (PCL). As these materials degrade, new living, autologous, tissue replaces the polymers. This neo-tissue formed within the tubular body encapsulates the remainder of the biostable polymer component of the tubular body. For example, but not limitation, the biostable polymer may be polycarbonate urethane (PCU). This remaining biostable polymeric component has plastic properties and can accommodate the growth of the tissue (expansion of the tube diameter) by exhibiting permanent deformation. Thus, the initial diameter of the tubular body in some embodiments is 12 mm and the final diameter of the tubular body is 24 mm.

The valve is fabricated from biostable polymer. In some embodiments, the biostable polymer, e.g., PCU, for both the tubular body and the valve is used. Using the same biostable polymer provides a structural continuity and good adhesion between the valve and the tubular body components. Thus, the biostable polymer maintains the structural continuity between the tubular body and the valve components. By using this configuration, the connection between the valve and the tubular body is mechanically robust.

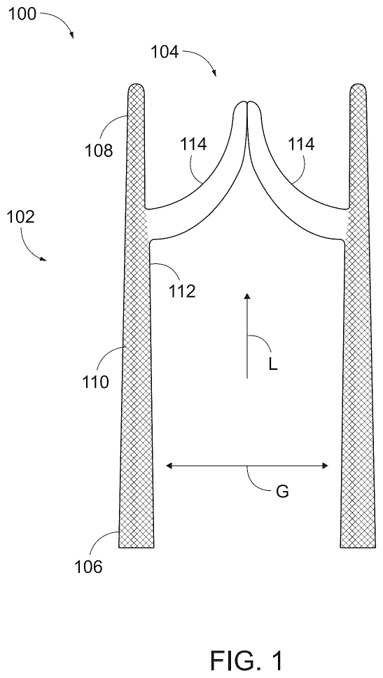

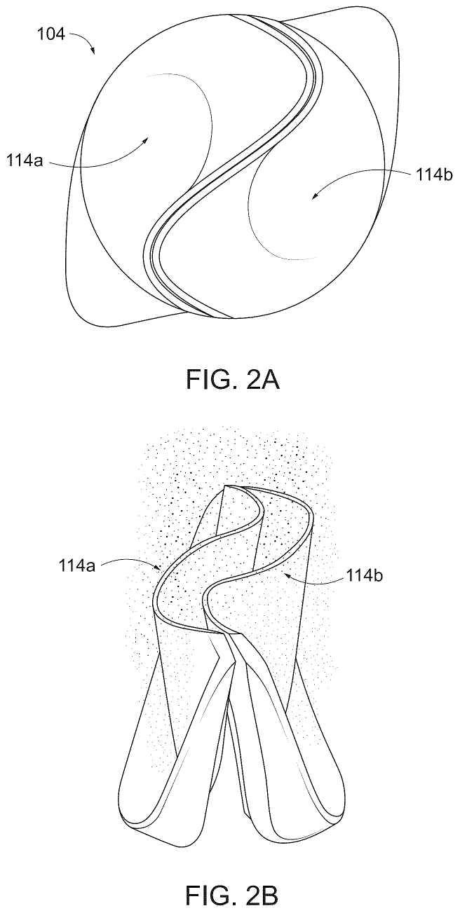

Referring to FIG. 1, a heart valve replacement 100 is provided. The heart valve replacement comprises a tubular body portion 102 having an inflow end 106, an outflow end 108 and a central portion 110 arranged between said inflow and outflow ends, defining a longitudinal direction L of the valve replacement and having an inner wall region 112. A valve 104 defined by at least one leaflet 114 is attached to the inner wall region 112 of the central portion 110 of the tubular body 100. Each leaflet 114 is movable between a closing position and an opening position such that the valve opens and closes. FIG. 2A shows a top perspective of the heart valve replacement 100 of FIG. 1. As shown the valve 104 includes first and second leaflets (114a and 114b) in a closed orientation, i.e., the free edges of leaflets 114a and 114b contact each other to form an inter-engagement that maintains the valve in the closed position. Referring to FIG. 2B, the first and second leaflets 114a and 114b are separable to provide an open position allowing the flow of blood through the valve 104.

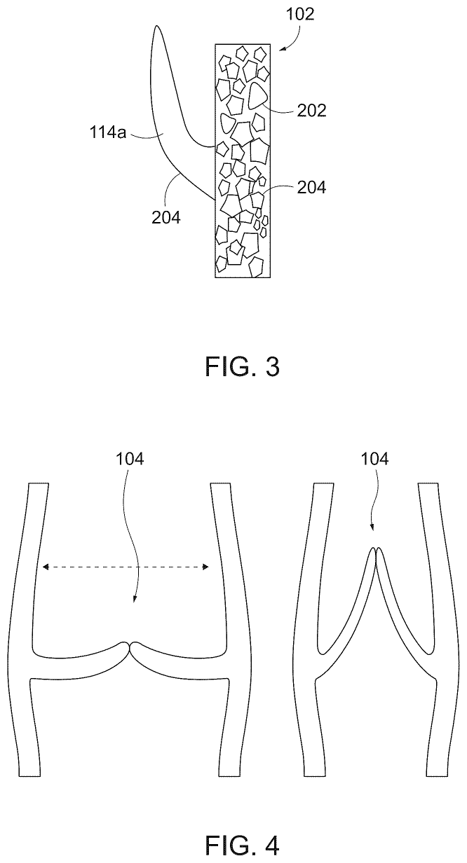

Referring to FIG. 3, in some embodiments, the tubular body portion 102 is fabricated from a combination of a biostable polymer 204 and a biodegradable biomaterial 202 adapted to allow in-growth of tissue of the host and to increase the replacement concomitantly with surrounding organ structures of a host. As shown in FIG. 1, growth of the tubular body or increase in diameter, is indicated by arrow G. Referring to FIG. 3, the valve leaflet(s) 114 is fabricated of also biostable polymer 204 and is connected to the biostable polymer 204 of the tubular body portion 102.

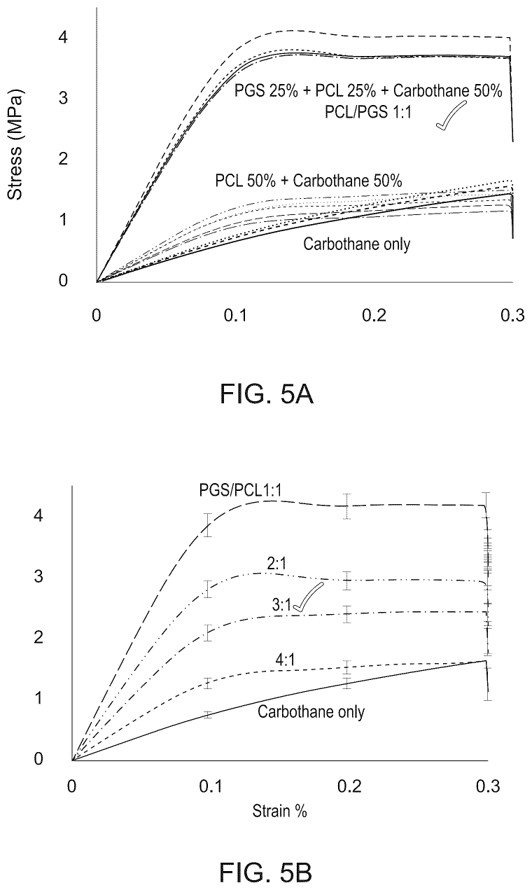

Referring to FIG. 4, the valve leaflets 114 have specific design features that promote a growth compatible valve such that the competency of the valve is maintained during expansion of the prosthetic as the child grows. For example, the leaflets are designed to have increased length, high coaptation length, and/or an increased length of free edge to account for the circumferential growth of the tube. These design features keep the valve competent despite the growth of the tube. As the tube expands circumferentially, the valve flattens but remains competent to close the valve.

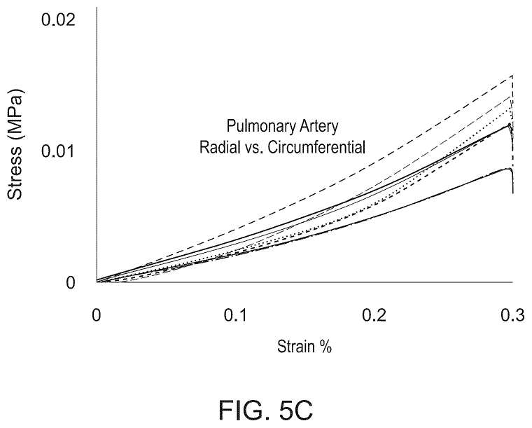

In one embodiment, the heart valve component comprises a tubular body including a combination of PCU, PGS and PCL and a valve comprising PCU. Referring to FIG. 5B, in some embodiments, the PGS:PCL ratio may be between about 1:1 to 4:1. The stress strain curve of the PGS:PCL and a PCU, i.e., Carbothane® only is shown in FIG. 5B. In one preferred embodiment, the ratio of PGS:PCL is about 3:1. Referring to FIG. 5A, the tubular body comprises in one embodiment, about 50 weight % polycarbonate urethane, 25 weight % PGS and 25 weight % PCL. In other embodiments, the tubular body comprises about 50 weight % polycarbonate urethane, 37.5 weight % PGS and 12.5 weight % PCL. Alternatively, the tubular body may include 50% weight % of PCU and 50 weight % PGS depending on the application for the heart valve replacement. In each of these embodiments, the degradation of the PGS and/or PCL cause an increase in porosity that will be replaced by living autologous tissue over time.

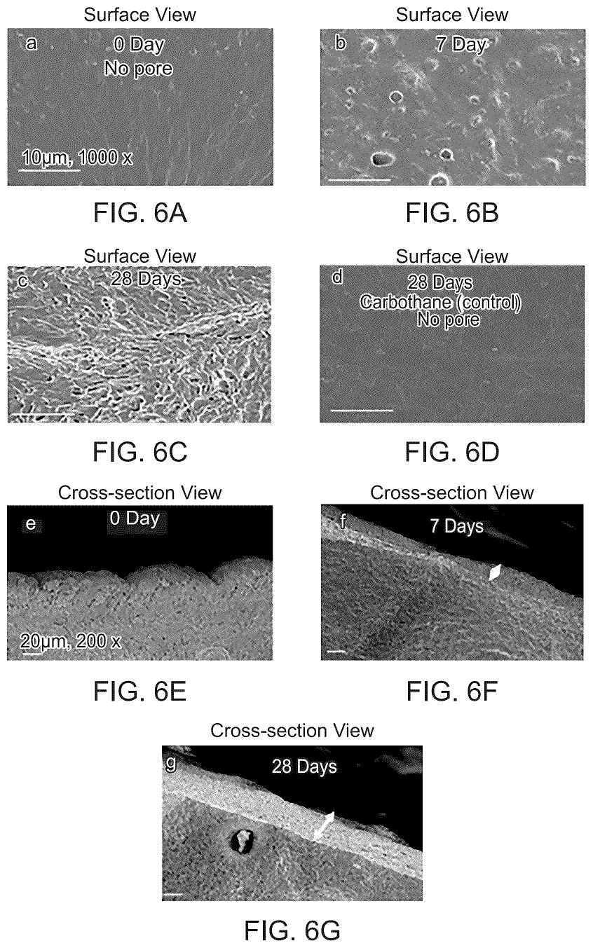

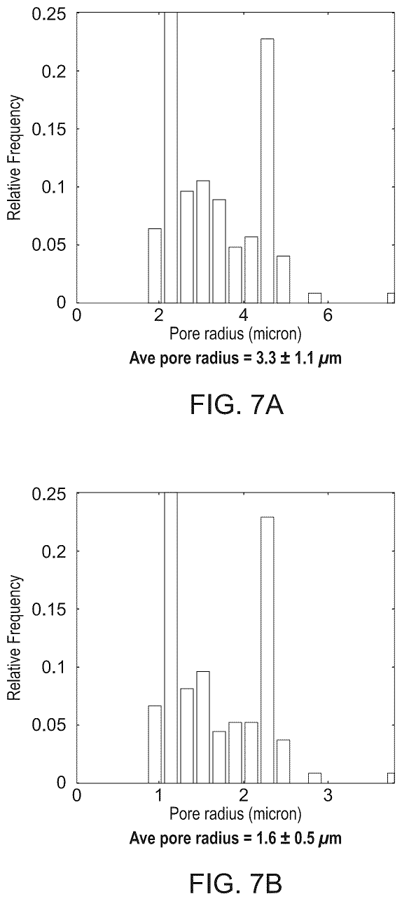

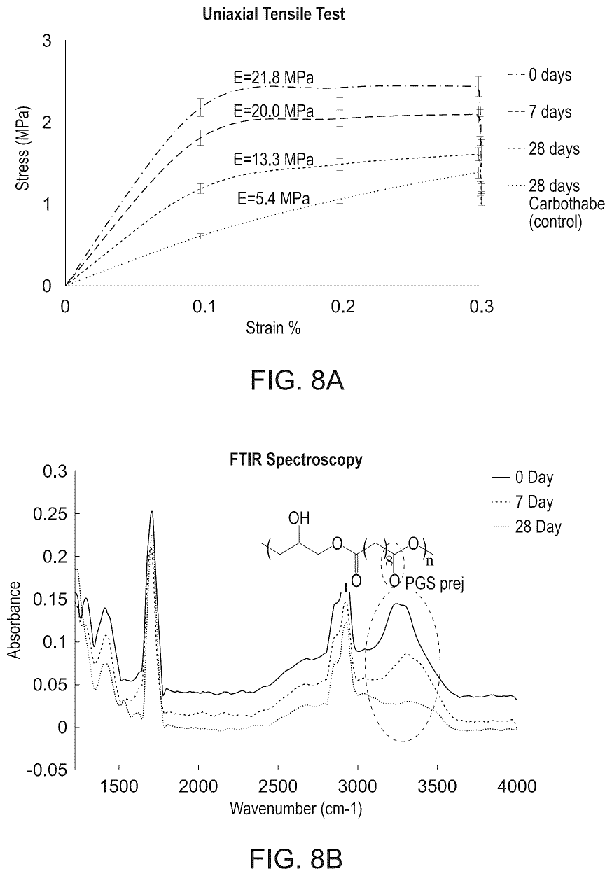

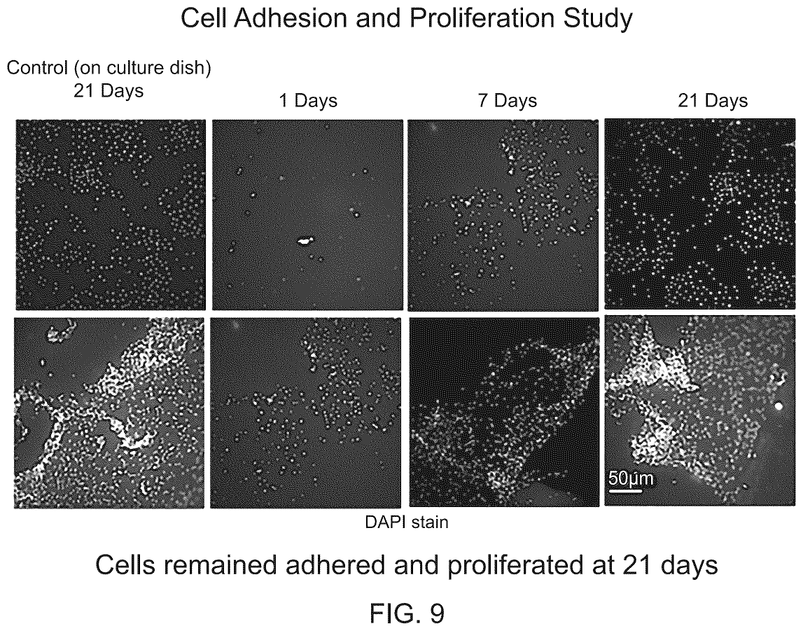

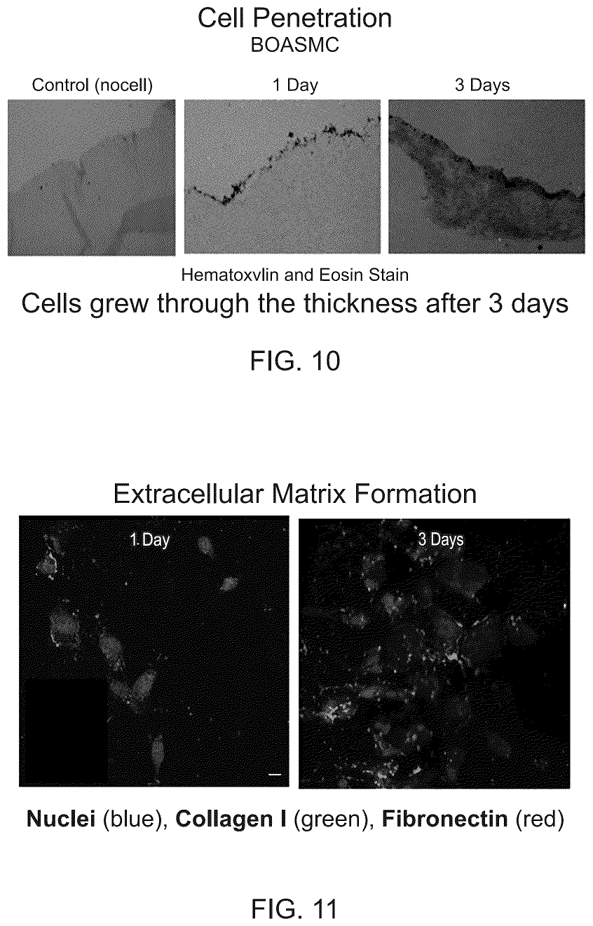

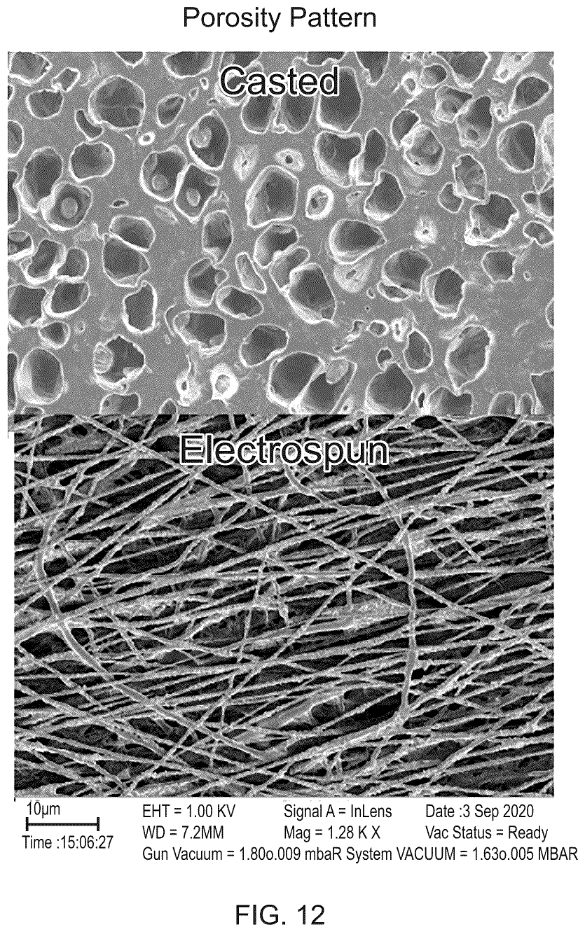

Referring to FIGS. 6A to 6G, scans of the surface view and cross section view of the tubular body during a degradation study is provided. As shown in FIGS. 6A to 6C, the biodegradable polymers in the tubular body degrade over time leading to surface porosity increases as a matter of degradation time, i.e., 0 days with no pores and 28 days with a significant porosity pattern. The pores can also be seen to increase at the cross sectional view of FIGS. 6E to 6G. As shown in the electron micrograph of FIG. 6D, the PCU control remained non-porous after 28 days. Thus, the tubular body comprises non-porous and porous sections over time. Referring to FIGS. 7A and 7B, the average pore radius at day 7 is 3.3±1.1 micron, whereas the average pore radius at day 28 is 1.6±0.1 micron. Referring to FIGS. 8A and 8B, uniaxial tensile test and FTIR spectroscopy show the elastic moduli and PGS decrease as a matter of degradation time of 0, 7 and 28 days. Thus, porosity of the tubular body increases while the biodegradable component of the tubular body decrease over degradation time to permitting in growth of live tissue in the porous structure. Referring to FIG. 9, a cell adhesion and proliferation study of an in vitro tubular body shows that living cells remain adhered to and proliferated at 21 days. FIG. 10 shows that the adhered cells grew after 3 days in a cell penetration study and FIG. 11 shows the secretion of extracellular matrix for smooth muscle cells in 3 days. Thus, the structure of the tubular body and its porosity pattern permits living tissue adhesion, proliferation, penetration and growth after implantation in the host.

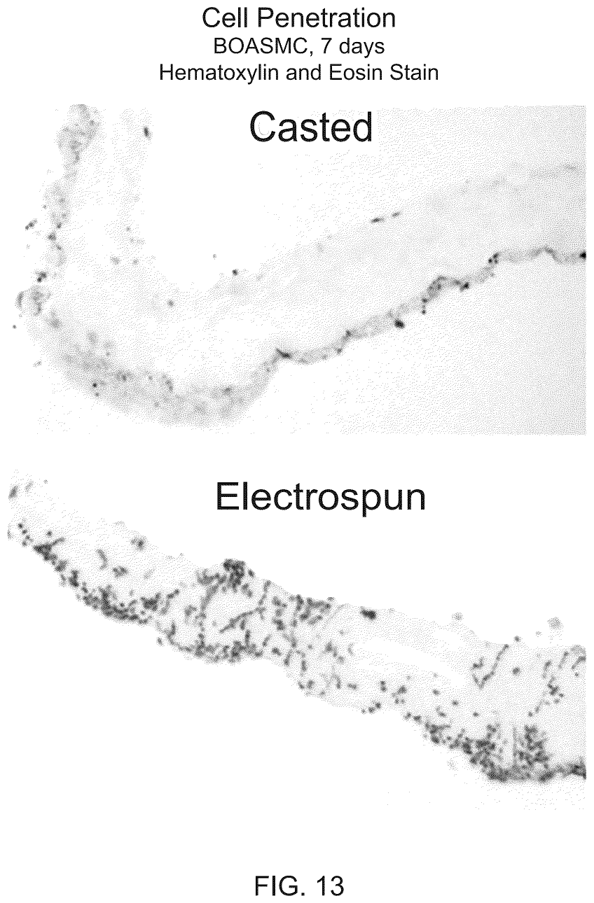

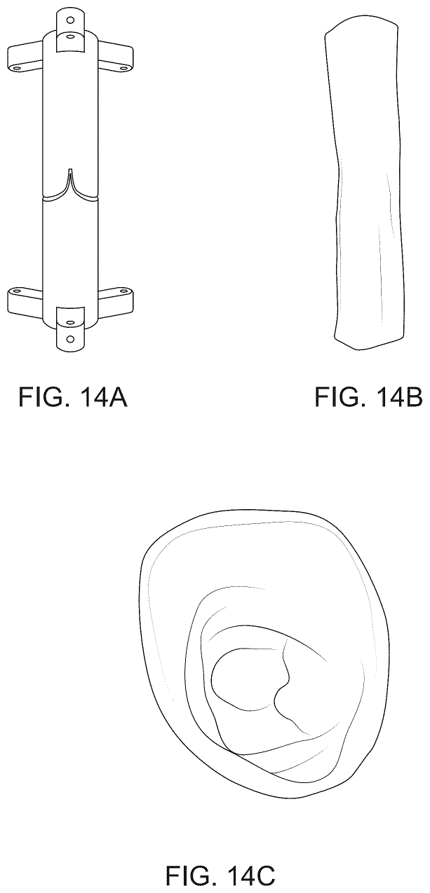

In some embodiments, the biostable and biodegradable polymers of the tubular body are electrospun fibers. It has been discovered that using electrospun fibers results in an interconnected porous network that provides a matrix that allows better replacement of degrading polymers with living tissue. Referring to FIG. 12, electron micrographs of the porosity pattern of a casted tubular body and an electrospun tublar body is shown. As depicted, the pores of the casted tubular body are individual circular pores, whereas the electrospun tubular body provides a larger surface area to volume ratio of interspaces resulting in increased porosity, as compared to the casted tubular body. This highly porous electrospun fiber network supports and guides cell growth and tissue regeneration, as shown in FIGS. 13. FIGS. 13 is a comparison of cell penetration in a casted tubular body as compared to an electrospun tubular body. As depicted, the electrospun body has a significantly higher cell penetration as compared to the casted body after 7 days.

In another aspect, a fabrication process is provided to manufacture the heart valve replacement described and embodied herein. In this regard, a valved tube is created, which comprises (1) a fully biostable valve that remains inert after implantation, (2) a porous tubular member formed from a mixture of biodegradable and biostable polymers, in which sections of the tubular member are replaced by autologous living and growing tissue over time after implantation, and (3) a mechanically robust cohesion provides a securement between the valve and tube.

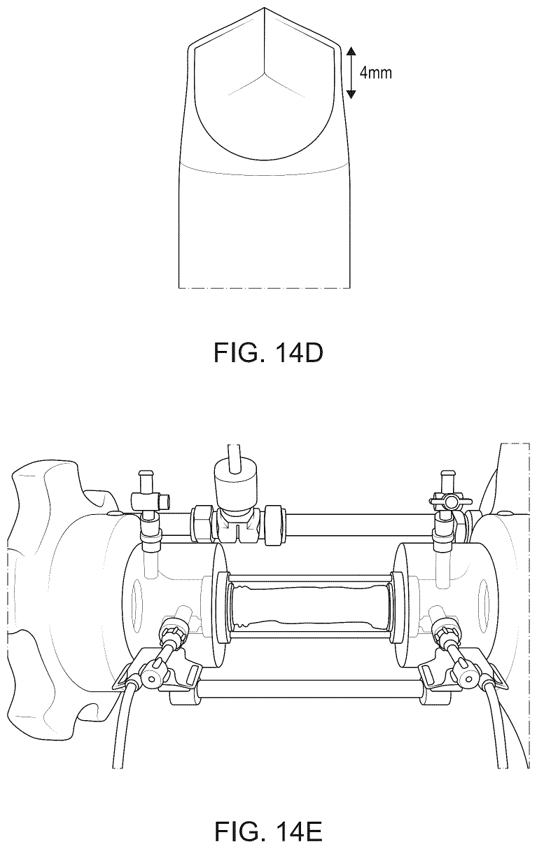

In one embodiment, a method of forming a heart valve replacement comprises preparing the valve using a mold, as shown in FIGS. 14A-14D. Then, the tube may be created by electrospinning on a rotating mandrel a polymeric mixture or solution capable of forming fibers. The term “electrospinning” or “electrostatic spinning” as used herein refers to a process in which fibers are formed from a solution or melt by streaming an electrically charged polymer solution or melt through an orifice. One advantages of using electrospun fibers in the tube of the heart valve replacement, is that very thin fibers can be produced having diameters, usually on the order of about 50 nanometers to about 25 microns, and more preferably, on the order of about 50 nanometers to about 5 microns. The polymeric electrospinning mixture or solution, for example, may be a combination PCU, PCL and PGS. Referring back to FIG. 12 (bottom), the electrospun fibers provide interstices or asymmetrical pores and high surface area per unit mass, resulting in a porous tube that permits replacement by autologous living and growing tissue over time after implantation. The mandrel may be round or it can be the shape of a predetermined blood vessel. As known by one of ordinary skill in the arts, the process described herein is exemplary and other processes may be used.

Other processes may include, for example, making the porous tube by lyophilization techniques. Some advantages of lyophilization include the ease of fabrication of the tube and control of its thickness. Knitting or braiding can be used to fabricate the porous tube. In this regard, the biodegradable and biostable polymer combination, can be processed as fibers via melt-spinning. Then the fibers can be further processed into a knitted tubular mesh. The advantages of knitting or braiding techniques are that the tube can be isotropic/anisotropic, and that various suitable biostable and biodegradable may be employed since most polymer resins can be melted and extruded as fibers. 3D printing techniques may also be used to fabricate the tube. 3D printing allows precise control over the macroscale properties, such as but not limited to curvature and bifurcations, and the microscale features such as porosity and surface roughness. Additionally, salt leaching may be used to fabricate the tube. In this regard, salt crystals with different sizes and different concentrations can be mixed in the polymeric composition. After the polymer dries, the salt is then leached out of the polymer by dissolving it in water, leaving behind the porous tube structure. The method for fabricating the porous tube may include any combination of two or more of these different fabrication processes. The importance of the tubular body for the heart valve replacement is the porosity of the structure to allow living tissue to grow into the structure, while also having non-porous sections to maintain the integrity and strength of the tubular body and attachment and securement of the valve component that is maintained despite degradation of the biodegradable component of tubular body. Other techniques to fabricate the valve, for example, include dip molding, such as injection molding, and/or 3D printing techniques.

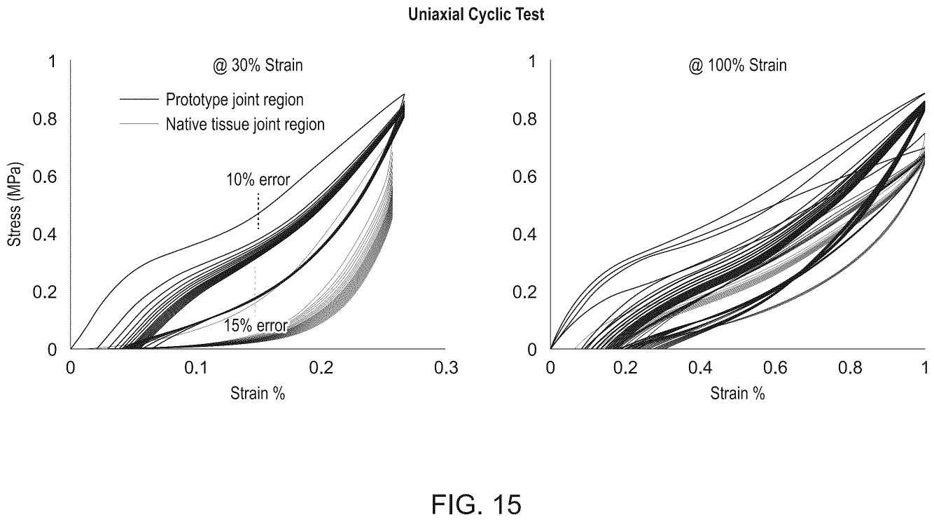

A mechanically robust cohesion between the valve and the tube that is maintained after degradation of the polymer forming the tube includes salt leaching to create porous tube walls that can fuse with the leaflets of the valve. The leaflet and the wall of the tube can be cast in one mold which allows the two polymer solutions to mix at the junction in between them. Both polymer solutions are soluble in a solvent, such as formaldehyde, and will therefore create a homogenous junction. Upon drying the polymers, the leaflet is fused to the wall of the tube, and the strength of the connection can be adjusted by increasing the contact area between the base of the leaflet and the wall. Other techniques for securing the valve to the wall of the tube include suturing, sintering, heat treatment, dip-coating the entire structure into a secondary hydrogel, and providing an outer layer of bioresorbable ring added to support the tube structure at the suture sights to maintain mechanical integrity. Referring to FIG. 15, the valve tube joint region may have sufficient strength such that no micro or macro signs of tear or fracture after 50 cycles at 30% or 100% strain on the region is apparent from a uniaxial cycle test.

In one embodiment, for example, dip-molding is used to make the heart valve replacement prosthesis. A monobloc fabrication method provides direct continuity between the biostable polymeric valve and the tube. It also can be used when it is desired to prevent the formation of an internal weak region by avoiding suturing and gluing. In some embodiments, the device is reinforced with a textile or electrospun layer to ensure additional strength for the valve-tube connection.

The replacement can be fabricated without sutures. Without sutures, the fabrication process is not human dependent, resulting in better reproducibility and lower costs of production. Further, the replacement can be manufactured with existing industrial fabrication techniques, which also provides better reproducibility. In addition, there are no suture holes, and therefore no hemostasis issues at the junction of tube/valve.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments and/or implementations can be understood and effected by those skilled in the art in practicing the claimed disclosure, from a study of the drawings, the disclosure and the appended claims.

What is claimed is:

1. A heart valve replacement comprising:
a tubular body having an inflow end, and outflow end and a generally cylindrical inner side wall portion extending between the inflow end and outflow end thereby forming a blood passage with a diameter, and
a valve defined by at least two leaflets, wherein each leaflets comprises first and second opposing portions and a longitudinal body therebetween, such that the first portion is secured to the inner side wall portion of said tubular body and the second portion is a free edge configured to engage corresponding second portion of an adjacent leaflet to close the valve, the inter engaging portions of the leaflets being separable to open the valve,
wherein the tubular body is composed of electrospun fibers, and further wherein the electrospun fibers include biostable polymeric fibers and biodegradable polymeric fibers.

2. The heart valve replacement of claim 1, wherein the tubular body permits in-situ tissue regeneration such that the diameter of the tubular member increases over time after implantation.

3. The heart valve replacement of claim 1, wherein the biodegradable polymeric fibers comprise at least one polymer selected from the group consisting of: polycapriolactone (PCL) and polyglycerol sebacate (PGS), or a combination thereof

4. The heart valve replacement of claim 3, wherein the biodegradable fibers are PGS and PCL, and further wherein the PGS:PCL ratio is between about 1:1 to 4:1.

5. The heart valve replacement of claim 4, wherein the ratio of PGS:PCL is about 3:1.

6. The heart valve replacement of any one of claims 1, wherein the biostable material is poly carbonate urethane (“PCU”).

7. The heart valve replacement of claim 1, wherein the tubular body comprises electrospun fibers of about 50 weight % polycarbonate urethane, 25 weight % PGS and 25 weight % PCL.

8. The heart valve replacement of claim 1, wherein the valve is sintered to the inner wall of the tubular body.

9. The heart valve replacement of claim 1, wherein the valve is attached to a portion of the tubular body formed only by biostable material.

10. The heart valve replacement of claim 1, wherein the at least two leaflets each have sufficient height to maintain the competency of the valve while the diameter of the tubular body increases over time.

11. The heart valve replacement of claim 1, wherein the at least two leaflets each have a height greater than the diameter of the tubular body.

12. The heart valve replacement of claim 1, wherein the at least two leaflets each have sufficient height of coaptation to maintain competency of the valve while the diameter of the tubular body increases over time.

13. The heart valve replacement of claim 1, wherein the at least two leaflets each have sufficient length of the free edge to maintain competency of the valve while the diameter of the tubular body increases over time.

14. The heart valve replacement of claim 1, wherein the valve is formed entirely from biostable material.

15. The heart valve replacement of claim 14, wherein the valve is formed from PCU.

16. The heart valve replacement of claim 1, wherein the replacement has an initial diameter of about 12 mm and a final diameter of about 24 mm.

17. A heart valve replacement comprising:
a tubular body portion comprising an inflow end, an outflow end and a central portion arranged between said inflow and outflow ends, defining a longitudinal direction of the valve replacement and having an inner wall region;
a valve comprising at least one leaflet attached to the inner wall region of the central portion, each one of said leaflets being movable between a closing position and an opening position of the valve,
wherein the tubular body portion comprises a combination of a biostable polymer and a biodegradable polymer such that the tubular body is configured to allow in-growth of tissue of a host after implantation and to increase its diameter concomitantly with surrounding organ structures of the host,
wherein the valve comprises entirely biostable polymer and is secured to the biostable polymer of the tubular body portion.

18. The heart valve replacement of claim 17, wherein the biodegradable biomaterial of the tubular body portion comprises PGS:PCL in a ratio between about 1:1 to 4:1.

19. The heart valve replacement of claim 18, wherein the ratio of PGS:PCL is about 3:1.

20. The heart valve replacement of any one of claims 17, wherein the biostable polymer of the tubular body portion is poly carbonate urethane.

21. The heart valve replacement of claim 17, wherein the biostable polymer of the tubular body portion is poly carbonate urethane and the biodegradable biomaterial of the tubular body is PGS and PCL.

22. The heart valve replacement of claim 21, wherein the tubular body portion comprises about 50 weight % polycarbonate urethane, 25 weight % PGS and 25 weight % PCL.

23. The heart valve replacement of claim 17, wherein the at least two leaflets each have sufficient height to maintain the competency of the valve while the diameter of the tubular body increases over time.

24. The heart valve replacement of claim 17, wherein the at least two leaflets each have a height greater than the diameter of the tubular body.

25. The heart valve replacement of claim 17, wherein the at least two leaflets each have sufficient height of coaptation to maintain competency of the valve while the diameter of the tubular body increases over time.

26. The heart valve replacement of claim 17, wherein the at least two leaflets each have sufficient length of the free edge to maintain competency of the valve while the diameter of the tubular body increases over time.

27. The heart valve replacement of claim 17, wherein the at least two leaflets are sintered to the inner wall of the tubular body.

28. The heart valve replacement of claim 17, wherein the valve is growth compatible.

29. The heart valve replacement of claim 17, wherein the combination of a combination of a biostable polymer and a biodegradable polymer of the tubular body portion are electrospun fibers.

30. The heart valve replacement of claim 29, wherein the valve does not include electrospun fibers.

31. A heart valve replacement comprising a porous electrospun tube comprising PCU, PGS and PCL.

32. The heart valve replacement of claim 31, wherein the PGS and PCL are in a ratio of between about 1:1 to 4:1.

33. The heart valve replacement of claim 32, wherein the ratio of PGS:PCL is about 3:1

34. The heart valve replacement of claim 31, wherein the tube comprises about 50 weight % polycarbonate urethane, 25 weight % PGS and 25 weight % PCL.

35. A method of fabricating a heart valve replacement device, the method comprising:
preparing a valve comprising a first biostable polymer on a mandrel,
preparing an electrospinning mixture the first biostable polymer and biodegradable polymers, and
electrospinning the electrospinning mixture of polymers onto the mandrel to form an interconnected porous tubular body, such that there is continuity between the first biostable polymers present in the valve and the tubular body.

36. The method of claim 35, wherein the biostable polymer is PCU and the biodegradable polymers are PGS and PCL.

37. The method of claim 35, wherein the valve is sintered to the inner wall of the tubular body portion.

38. The method of claim 36, wherein weight ratio of PCU is about 50% per total weight of the electrospinning mixture.

39. The method of claim 36, wherein the weight ratio of PGS is about 25% per total weight of the electrospinning mixture.

40. The method of claim 36, wherein the weight ratio of PCL is about 25% per total weight of the electrospinning mixture.

41. The method of claim 36, wherein the PGS and PCL are in a ratio of between about 1:1 to 4:1.

42. The heart valve replacement of claim 41, wherein the ratio of PGS:PCL is about 3:1.

43. The heart valve replacement of claim 35, wherein the valve is formed from dip molding or 3-D printing techniques on the mandrel.

44. The heart valve replacement of claim 35, wherein the tubular body comprises electrospun fibers of about 50 weight % PCU, 25 weight % PGS and 25 weight % PCL.

45. A method of replacing a heart valve in a host, comprising the steps of:
inserting a distal end portion of a delivery sheath into a portion of a heart of a host,
the delivery sheath having a heart valve replacement according to any one of claims 1 to 36 disposed within a lumen of the delivery sheath,
moving the heart valve replacement distally out of the delivery sheath; and
positioning the heart valve replacement within the heart of the host.

46. The method of claim 45, wherein the method is a method is a method for treating the host for aortic stenosis, mitral valve stenosis, regurgitation, or tricuspid valve regurgitation.

47. The method of claim 45, wherein the host is a child under the age of eighteen years old.

48. The method of claim 47, wherein the living tissue of the child replaces a portion of the heart valve replacement over time.

49. A heart valve replacement comprising:
a tubular body portion comprising an inflow end, an outflow end and a central portion arranged between said inflow and outflow ends, defining a longitudinal direction of the valve replacement and having an inner wall region;
a valve comprising at least one leaflet attached to the inner wall region of the central portion, each one of said leaflets being movable between a closing position and an opening position of the valve,
wherein the tubular body portion comprises a combination of a biostable polymer and a biodegradable polymer such that the tubular body is configured to allow in-growth of tissue of a host after implantation and to increase its diameter concomitantly with surrounding organ structures of the host,
wherein the valve comprises entirely biostable polymer and is secured to the biostable polymer of the tubular body portion, and further wherein the replacement is manufactured by one or more of the processes selected from the group consisting of: lyophilization, knitting, braiding, 3D printing, and a combination thereof.
